(19) United States
(12) Patent Application Publication
(10) Pub. No.: US 2012/0058204 A1
(43) Pub. Date: Mar. 8, 2012

(54) USE OF OVER EXPRESSION OF CYSTATHIONINE GAMMA LYASE AS A PROGNOSTIC, DIAGNOSTIC AND THERAPEUTIC TARGET FOR CANCER

(75) Inventors: Tesshi Yamada, Tokyo (JP); Kazufumi Honda, Tokyo (JP); Wilber Huang, Taipei City (TW)

(73) Assignees: NATIONAL CANCER CENTER RESEARCH INSTITUTE, Tokyo (JP); ABNOVA CORPORATION, Taipei (TW)

(21) Appl. No.: 13/226,730

(22) Filed: Sep. 7, 2011

Related U.S. Application Data
(60) Provisional application No. 61/380,475, filed on Sep. 7, 2010.

(51) Int. Cl. A61K 33/24; A61P 35/00; A61K 38/05; C40B 30/04; C12Q 1/527; G01N 33/53

(52) U.S. Cl. 424/649; 435/4; 435/7.1; 435/7.92; 514/19.3; 506/9

(57) ABSTRACT

The present invention relates to a method of identifying an expression level of cystathionine gamma lyase (CTH) in a sample from a subject. The present invention further discloses a method of diagnosing a subject with cancer having risk of resistance to a platinum-based drug. The present invention also discloses a method for improving efficacy of a platinum-based drug in a subject suffering a cancer.

### FIELD OF THE INVENTION

The present invention relates to a method for identifying an expression level of cystathionine gamma lyase (CTH) in a sample from a subject. The present invention also relates to a method for diagnosing a subject with cancer having risk of resistance to a platinum-based drug. The present invention further relates to a method for improving efficacy of a platinum-based drug in a subject suffering a cancer.

### BACKGROUND OF THE INVENTION

Ovarian cancer is the second most common gynaecological cancer worldwide and the sixth most common cancer in women overall. The five-year survival rates for the various clinical stages of the disease are as follows: Stage I>90%, Stage II=80%, Stage III=20% and Stage IV=10%; there is a significant drop in the survival rates at later stages of the disease. Standard-of-care treatment for advanced stages of the disease includes cytoreductive surgery followed by chemotherapy.

For most patients there is a low probability of surviving, since approximately 75% of all patients are diagnosed at stage III and IV of the disease, and poor prognosis is associated with late diagnosis of the disease at its advanced stages. In addition, resistance to currently-available chemotherapeutic agents is another major problem. For example, epithelial ovarian cancer, the most common type of ovarian cancer, is one of the most platinum-sensitive solid malignancies, with 70% of patients achieving a complete clinical remission after front-line therapy with a platinum-based chemotherapeutic regimen. However, despite this initial success, approximately 50% of patients will develop recurrent disease within 3 years of diagnosis. Paradoxically, although most patients initially respond to platinum chemotherapy, the majority eventually die from chemotherapeutic resistant disease. The identification of a potential target related to chemotherapeutic resistance could represent a significant advancement in our ability to treat these often fatal malignancies. Moreover, the detailed knowledge about the drug resistance status of a give patient with cancer can provide the basis for an individual patient-tailored chemotherapy regiment in the future. To achieve this, an exact prediction of the resistance status of a tumor patient is necessary.

### BRIEF DESCRIPTION OF THE DRAWINGS

FIG. 1 shows profiling of protein expression obtained from virtual pathology. (A) Diagram of quantitative virtual immunofluorescent microscopy (AQVIP) algorithm; (B) a virtual pathology image constructed from immunofluorescent microscopy of anti-CK19 antibody (green) and target antibody (red) double staining on tissue microarray (TMA) of ovarian cancer; (C) the epithelial cells were proven with anti-CK19 antibody (green); (D) image binarization of epithelial cells (white); (E) high resolution photograph in image binarization of epithelial cells; and (F) high resolution virtual pathology image for expression of target antigen (red). Expression of target antigen is automatically calculated from total area in tumor into total fluorescence intensity. This numerical value was defined as AQVIP score.

FIG. 2 shows immunofluorescent microscopy images of virtual pathology with immunohistochemistry for CTH on TMA samples (done by training cohort-NDMC). Green color represents CK19 and red color represents CTH. Scores were calculated through AQVIP. The bright field images were shown on the right column.

FIG. 3 shows Kaplan-Meier analysis of CTH by National Defense Medical College cohort (training-cohort-NDMC) and National Cancer Center cohort (validation-cohort NCC) through AQVIP. Upper columns: Kaplan-Meier analysis of overall survival. Red lines represent cases with up-regulation of protein expression. Blue lines represent cases with down-regulation of protein expression. Lower columns: the frequency distribution of intensity scores of each analysis. Red color bars represent positive cases. Blue color bars represent negative cases. X-axis is intensity score and Y-axis is number of cases. Arrow heads indicate cut-off values.

FIG. 4 shows Cox proportional hazard model of CTH. Protein expression was calculated with univariate hazard ratio and multivariate hazard ratio. X-axis is hazard ratio. Circles are hazard ratio for death of tumor. Bars represent 95% confidence intervals. Black bars means without significance, and red bars means statistically significant.

FIG. 5 shows predictive biomarkers for chemosensitivity of ovarian cancer in advanced-stage. (A) Correlation between CTH protein expression and chemosensitivity of ovarian cancer in advanced-stage. (B-C) Representative results of immunohistochemistry for CTH expression in regular pathological sections from strongly positive cases (B) and negative cases (C). (D) Kaplan-Meier analysis of overall survival for ovarian cancer in advanced-stage. The samples were evaluated through immunohistochemistry using DAB by National Defense Medical College cohort (Tokorozawa, Japan). (E) CTH expression in ovarian cancer cell lines. (F-K) In vitro effect of combined treatment with propargylglycine (PPG, specific inhibitor of CTH) and cysplatin (CDDP) on ovarian cancer cell lines. OVISE, OVTOKO and OVCAR3 were cultured with various concentration of PPG and IC50s were measured. PPG alone did not affect cell growth in the concentration ranging from 0.1 g/ml to 10 g/ml. (F-H). These cell lines were also cultured in the culture media containing 10 g/ml PPG and various concentration of CDDP or containing CDDP alone. Compared with cells cultured in medium without PPG, the IC50s of those cultured in the PPG-containing medium (10 g/ml) decreased (I-K).

FIG. 6 shows correlation between protein expression and AQVIP scores. Pathologist blindly observed the regular pathological sections obtained from National Defense Medical College (n=132). Expression level of CTH was evaluated and classified into six categories. Category 1: staining in the cytoplasm of cancer cells is not observed (negative). Category 2: staining in the cytoplasm of cancer cell is weaker than that of smooth muscle (weakly positive). Category 3: a situation between category 2 and category 4 (weakly positive). Category 4: staining in the cytoplasm of cancer cell is equivalent to smooth muscle (weakly positive). Category 5: staining of cancer cells is stronger than that of smooth muscle. However, the percentage of cells fitted into category 5 is less than 30% in cancer area (weakly positive).

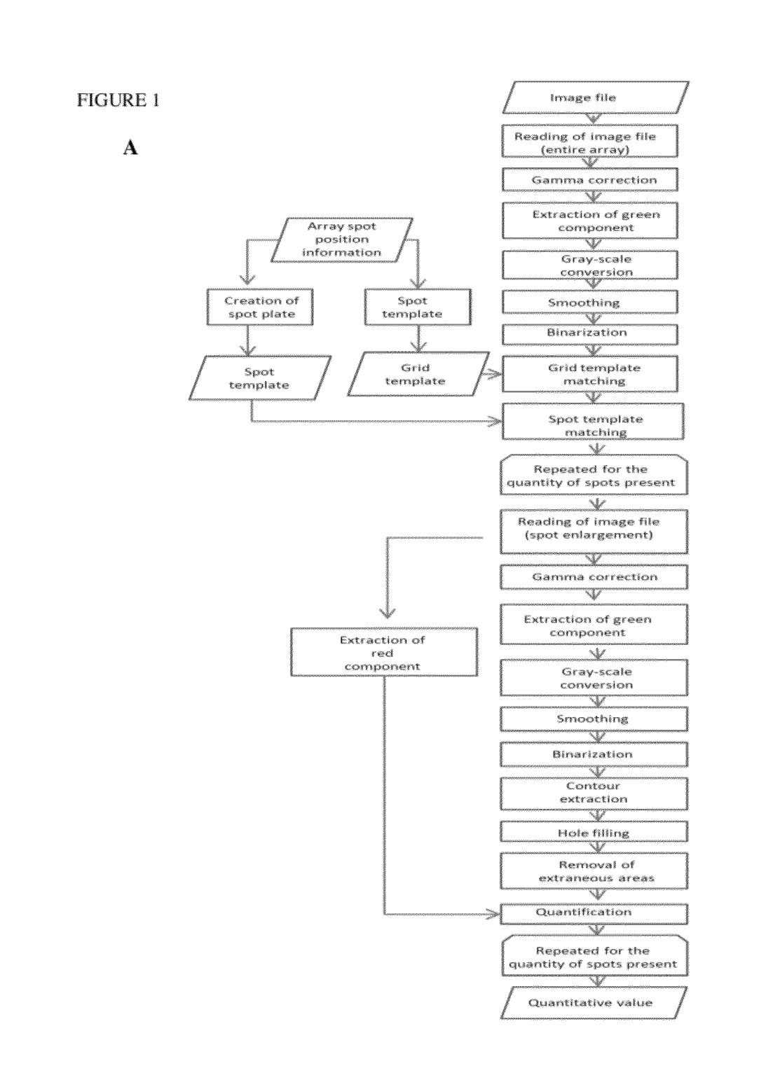

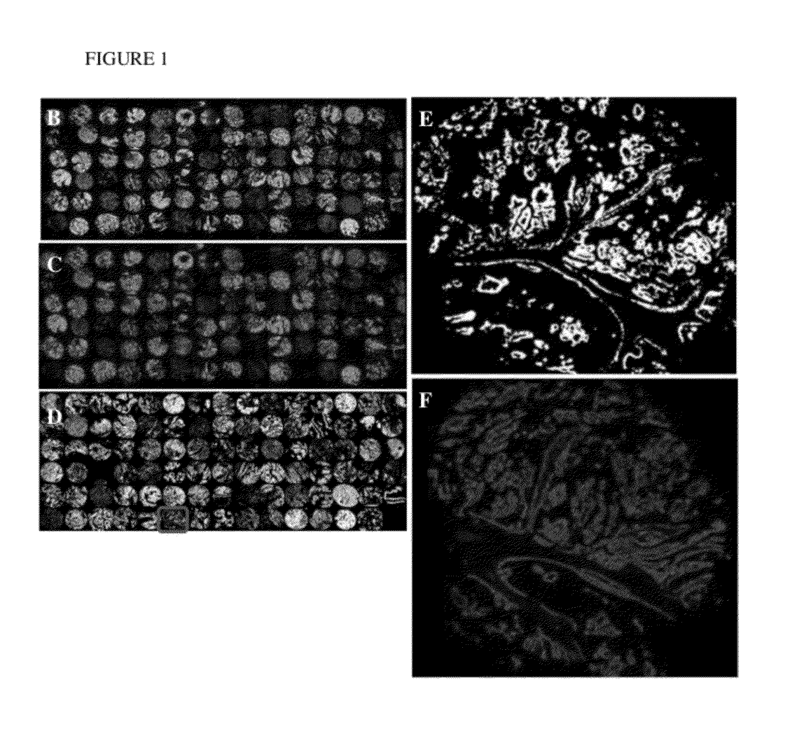

### SUMMARY OF THE INVENTION

The present invention discloses a method of identifying an expression level of cystathionine gamma lyase (CTH) in a sample from a subject comprising: (a) determining the normal expression level of CTH from a normal subject; (b) obtaining the sample from the subject; and (c) determining the expression level of CTH from the sample as over expression if the expression level of CTH from the sample is higher than the normal expression level of CTH; or the expression level of CTH from the sample as normal expression if the expression level of CTH from the sample is equal to the normal expression level of CTH.

The present invention further discloses a method of diagnosing a subject with cancer having risk of resistance to a platinum-based drug comprising: (a) identifying an expression level of CTH in a sample from the subject with cancer by the method of identifying an expression level of cystathionine gamma lyase (CTH) in a sample from a subject comprising: (i) determining the normal expression level of CTH from a normal subject; (ii) obtaining the sample from the subject; and (iii) determining the expression level of CTH from the sample as over expression if the expression level of CTH from the sample is higher than the normal expression level of CTH; or the expression level of CTH from the sample as normal expression if the expression level of CTH from the sample is equal to the normal expression level of CTH; and (b) determining the subject having risk of resistance to a platinum-based drug if the sample is CTH over expression.

The present invention also discloses a method for improving efficacy of a platinum-based drug in a subject suffering a cancer comprising: (a) identifying an expression level of CTH in a sample from the subject with cancer by the method of identifying an expression level of cystathionine gamma lyase (CTH) in a sample from a subject comprising: (i) determining the normal expression level of CTH from a normal subject; (ii) obtaining the sample from the subject; and (iii) determining the expression level of CTH from the sample as over expression if the expression level of CTH from the sample is higher than the normal expression level of CTH; or the expression level of CTH from the sample as normal expression if the expression level; and (b) administering a CTH inhibitor to the subject having risk of resistance to a platinum-based drug if the sample is CTH over expression.

### DETAILED DESCRIPTION OF THE INVENTION

In the present invention, the inventors firstly developed the high-throughput quantitative system to analyze the protein expression from virtual pathology images. This system had the performance which could scan a TMA stained with double immunofluorecsence in 25 minutes, and therefore was able to scan about 50 slides in 24 hours. The inventors have used this system and screened antibodies to discover the novel biomarker for prediction of clinical outcome of a cancer (such as ovarian cancer).

The benefit of this system is to be able to automatically calculate the average intensity of immunofluorescence in all cancer area. Therefore, this system possesses a high-throughput potential for proteome-scale antibody screening. After construction of huge images of virtual pathology with double immunofluorescence, each image of virtual pathology, quantitative expression of antigen and scrambled clinical findings were combined and a database for antibody-based proteomics is constructed.

A new bioinformatics tool, X-tile algorithm, was used to select potential biomarkers from the expression profile. The inventors calculated the cut-off point of each antibody and classify the antibodies into positive and negative group by using of X-tile algorithm. In order to validate the biomarkers evaluated by the bioinfomatics, the validation assay was performed by another independent cohort which was obtained by other medical institute when it was different. Patients with CTH overexpression demonstrated worse prognosis than those without that. According to Cox-multivaliate analysis, CTH expression was an independent prognostic factor. The predictive biomarker was searched based on the results of the first CDDP-based chemotherapy. CTH demonstrated significant differences in expression profile between responder and non-responder of first CDDP-based chemotherapy. CDDP has been developed as drug of molecular target therapy, the inhibitor of protein overexpressed in CDDP-based chemotherapy non-responders therefore could be the candidate of target therapy. CTH was selected as molecular target therapy candidate of ovarian cancer for several reasons: 1) CTH expression is increased in patients with poor prognosis of ovarian cancer; 2) CTH is an independent risk factor for death in ovarian cancer; and 3) there has correlation between CTH expression and response of first CDDP-based chemotherapy. CTH is an enzyme that catalyzes the convention from cystahionine to cysteine, which is further metabolized into glutathione (GSH) and metallothionein (MT). It has been known that CDDP exposure induces MT and GSH in vivo and in vitro. It was reported that MT and GSH make the cancer cells resistant to cisplatin by metabolizing the drug into inactive form. As an irreversible inhibitor of CTH, propargylglycine decreases the intracellular pool of free cystein and thereby decreases synthesis of MT and GSH. Overexpression of CTH is only detected in about 22% of tested ovarian cancer cell lines (two out of nine) Combination of propargylglycine and CDDP is more effective in inhibiting growth of CTH-overexpressed tumor than each one alone. Therefore, protein expression of CTH in pathological samples could be a predictive biomarker of CDDP-based chemotherapy efficacy. Moreover, combination of propargylglycine and CDDP may be a novel molecular target therapy for patients resistant to CDDP-based chemotherapy.

For clinical application of CTH as an ovarian cancer biomarker, general immunohistochemical analysis using DAB is also employed to evaluate the protein expression. Strongly positive cases defined by immunohistochemistory has significantly poorer prognosis than weakly positive and negative cases. In addition, expression of CTH is frequently detected in clear cell adenocarcinoma in comparison with other types of ovarian cancer. It is well known that clear cell adenocarcinoma of ovary is chemoresistant against CDDP. CTH overexpression could be one of the factors contributed to the chemoresistance.

Accordingly, the present invention provides a method of identifying an expression level of cystathionine gamma lyase (CTH) in a sample from a subject comprising: (a) determining the normal expression level of CTH from a normal subject; (b) obtaining the sample from the subject; and (c) determining the expression level of CTH from the sample as over expression if the expression level of CTH from the sample is higher than the normal expression level of CTH; or the expression level of CTH from the sample as normal expression if the expression level of CTH from the sample is equal to the normal expression level of CTH. The prognosis is indicated as positive if the sample is CTH over expression.

As used herein, the term “subject” is intended to include human and non-human animals. The term “normal subject” refers to a subject who does not suffer from the cancerous disease being tested for.

In one embodiment, the method is employed in the normal subject.

In another embodiment, the method is employed in the subject suffers pancreatic, bladder, renal, prostate or ovarian cancer. Preferably, the cancer is ovarian cancer.

In yet another embodiment, the method is employed in the subject having the first biopsy for suspicion of cancer, or being prognosed before treated with a platinum-based drug or after treated with a platinum-based drug.

The term “sample” refers to a sample of a body fluid, to a sample of separated cells or to a sample from a tissue or an organ. Samples of body fluids can be obtained by well known techniques and include, preferably, samples of blood, plasma, serum, or urine, more preferably, samples of blood, plasma or serum. Tissue or organ samples may be obtained from any tissue or organ by, e.g., biopsy. Separated cells could be obtained from the body fluids or the tissues or organs by separating techniques such as centrifugation or cell sorting.

The term “immunoassay” refers to any assay that utilizes an antibody to specifically bind a target protein. In the present invention, the anti-CTH monoclonal or polyclonal antibody is used. Examples of immunoassays include, but are not limited to, western blotting, single-antibody or double-antibody sandwich enzyme-linked immunosorbent assay (ELISA), immunohistochemistry (IHC), immunocytochemistry (ICC) or immunofluorescence (IF). In an ELISA or EIA assay, a label enzyme and substrate are used to produce an amplified signal.

The term “antibody” used herein refers to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies (e.g., bispecific antibodies). The term “antibody” also includes antigen binding forms of antibodies, including fragments with antigen-binding capability (e.g., Fab′, F(ab′)₂, Fab, Fv and rIgG). The term also refers to recombinant single chain Fv fragments (scFv). The term antibody also includes bivalent or bispecific molecules, diabodies, triabodies, and tetrabodies. The term also refers to subhuman primate antibody, murine antibody, rabbit antibody, chicken antibody, chimeric antibody, phage antibody, humanized antibody, or human antibody.

The term “prognosis” refers to a prediction of medical outcome, for example, a poor or good outcome (e.g., likelihood of long-term survival); a negative prognosis, or poor outcome, includes a prediction of relapse, disease progression (e.g., tumor growth or metastasis, or drug resistance), or mortality; a positive prognosis, or good outcome, includes a prediction of disease remission, (e.g., disease-free status), amelioration (e.g., tumor regression), or stabilization.

The term “CTH” used herein refers to cystathionine gamma lyase or cystathionase, Entrez GeneID: 1491.

The present invention further discloses a method of diagnosing a subject with cancer and without being administered with the platinum-based drug having risk of resistance to a platinum-based drug comprising: (a) identifying an expression level of CTH in a sample from the subject with cancer by the method of identifying an expression level of cystathionine gamma lyase (CTH) in a sample from a subject comprising: (i) determining the normal expression level of CTH from a normal subject; (ii) obtaining the sample from the subject; and (iii) determining the expression level of CTH from the sample as over expression if the expression level of CTH from the sample is higher than the normal expression level of CTH; or the expression level of CTH from the sample as normal expression if the expression level of CTH from the sample is equal to the normal expression level of CTH; and (b) determining the subject having risk of resistance to a platinum-based drug if the sample is CTH over expression. In one embodiment, the cancer is pancreatic, bladder, renal, prostate or ovarian cancer. Preferably, the cancer is ovarian cancer.

The term “platinum-based drug (or compound)” as used herein refers to a compound comprising a heavy metal complex containing a central atom of platinum surrounded by organic and/or inorganic functionalities. Non-limiting examples of platinum-based drugs include cisplatin, carboplatin, oxilaplatin, nedaplatin, spiroplatin, iproplatin, satraplatin, pharmaceutically acceptable salts thereof, stereoisomers thereof, derivatives thereof, analogs thereof, and combinations thereof.

The present invention also discloses a method for improving efficacy of a platinum-based drug in a subject suffering a cancer comprising: (a) identifying an expression level of CTH in a sample from the subject with cancer by the method of identifying an expression level of cystathionine gamma lyase (CTH) in a sample from a subject comprising: (i) determining the normal expression level of CTH from a normal subject; (ii) obtaining the sample from the subject; and (iii) determining the expression level of CTH from the sample as over expression if the expression level of CTH from the sample is higher than the normal expression level of CTH; or the expression level of CTH from the sample as normal expression if the expression level; and (b) administering a CTH inhibitor to the subject having risk of resistance to a platinum-based drug if the sample is CTH over expression. In one embodiment, the cancer is pancreatic, bladder, renal, prostate or ovarian cancer. Preferably, the cancer is ovarian cancer.

The term “CTH inhibitor” refers to a biochemical or chemical material or molecule which preferably inhibits or reduces enzymatic activity of CTH or the expression of the CTH gene or the localization of CTH in the cell. In one embodiment, the CTH inhibitor is D-propargyl glycine, L-propargyl glycine, DL-propargyl glycine, or salts thereof.

In one embodiment of the present invention, the CTH inhibitor is administered to the subject before, after, or concomitantly with the treatment of the platinum-based drug, wherein “concomitantly” means administering the agents substantially concurrently. The term “concomitantly administering” encompasses not only administering the two agents in a single pharmaceutical dosage form but also the administration of each active agent in its own separate pharmaceutical dosage formulation. Where separate dosage formulations are used, the agents can be administered at essentially the same time, i.e., concurrently.

### EXAMPLE

The examples below are non-limiting and are merely representative of various aspects and features of the present invention.

### Example 1

### Material and Methods

### Patients and Tissue Samples

This invention was performed with approval of the Internal Review Board on ethical issues of the National Defense Medical College Hospital (NDMC, Tokorozawa Japan) and National Cancer Center Hospital (NCC, Tokyo Japan). Two independent cohorts were prepared: the training cohort to identify predictive biomarkers of the clinical outcome of ovarian cancer in advance-stage, and the validation cohort to verify the results. Follow-up was calculated from the data of initial definitive surgery to the data of either last follow-up or death. None of the patients had undergone neo adjuvant chemotherapy or radiation therapy before surgery.

### Training Cohort-NDMC

The NDMC-training cohort consisted of 139 patients who underwent initial surgery followed by platinum-based chemotherapies for stage III or IV primary ovarian cancer at the Department of Obstetrics and Gynecology, NDMC, Tokorozawa, Japan, between 1987 and 2005. Formalin-fixed paraffin-embedded tissue samples were prepared at the Department of Laboratory Medicine. All of the pathology specimens were reviewed, and the histological tumor types were classified according to the WHO criterion. Histological grading, with reference to the grading system proposed by Shimizu et al. and Silverberg, was preformed as described previously (Shimizu Y et al., Cancer 1998; 82:893-901; Silverberg S G, Int J Gynecol Pathol 2000; 19:7-15; and Yamamoto S et al., Mod Pathol 2007; 20:1278-1285). The International Federation of Gynecology and Obstetrics (FIGO) system was used for assigning the stage of the tumors. The chemotherapeutic regimes comprised of cyclophosphamide (CPA), doxorubicin (DXR), and cisplatin (CDDP) in 80 patients; paclitaxel (PTX) and carboplatin (CBDCA) in 30; irinotecan (CPT-11) and CDDP in 8; etoposide (VP-16) and CDDP in 7; docetaxel (DOC) and CDDP in 6; CPA and CDDP in 3; CPT-11 and CBDCA in 1; and CPA and CDDP in 1. Clinical response to chemotherapy was evaluated by ultrasonography or computed tomography and classified into complete response (CR), partial response (PR), stable disease (SD), and progression disease (PD) according to the new Response Evaluation Criteria for Solid Tumors (RECIST) guidelines (Table 1).

TABLE 1

| | National | National |
|---|---|---|
| | Defense College | Cancer Center |
| | Case numbers | Case number |
| | (n = 139) | (n = 47) |
| Age | | |
| 56< | 79 | 22 |
| 55≧ | 60 | 25 |
| FIGO^{a} stage | | |
| III | 104 | 37 |
| IV | 35 | 10 |
| Residual tumor (cm) | | |
| 0-2 | 62 | 25 |
| 2< | 77 | 18 |
| Unkown | 0 | 4 |
| Histological grade^{b} | | |
| 1 or 2 | 42 | — |
| 3 | 97 | — |
| Histological type | | |
| Serus | 89 | 32 |
| Clear cell | 27 | 8 |
| Endometrioid | 10 | 3 |
| Mucinous | 13 | 3 |
| unknown or unclassified | 0 | 1 |
| Response to hemotherapies^{c} | | |
| Compleate response/partial | 44 | 11 |
| response | | |
| Stable disease/progressive | 33 | 3 |
| disease | | |
| unknown | 0 | 4 |

^{a}International Federation of Gynecology and Obsterics
^{b}Silberberg's grading
^{c}Patients with residual tumors >2 cm in size after initial surgery

### Validation Cohort-NCC

The NCC-validation cohort consisted of 47 patients with stage III or IV primary ovarian cancer who underwent surgery within the Gynecology Division, National Cancer Center Hospital (NCC), Tokyo, Japan between 1983 and 2001 (Table 1).

### Tissue Microarray (TMA)

To construct TMA blocks, formalin-fixed paraffin-embedded cancer tissue blocks from all cases that contained the areas that had been used for histological grading were selected. Specimens of 2.0 mm in diameter from each case were taken from these blocks and transferred to recipient blocks using a Tissue Microarrayer (Beecher Instrument, Silver Spring, Md., USA). These TMA blocks were then cut into 4-m-thick sections and subjected to immunofluorescent analyses and the construction of virtual pathology.

### Cell Lines

All ovarian cancer cell lines used in this study (OVISAHO, OVTOKO, OVISE, KURAMOCHI, RMUG-S, TYK-nu Cp-r, TYK-nu, ES-2, and OVACAR-3) were obtained from the American Type Culture Collection or the Japan Collection of Research Bioresources (Osaka, Japan). All ovarian cancer cells were cultured as recommended.

### Antibodies

A CTH mouse monoclonal antibodies were generated at Abnova (Abnova, Taipei, Taiwan, clone number MO3). Specificity of all antibodies to corresponding antigens was verified by western blotting, and by TMAs of normal human tissues at Abnova.

### Immunohistochemistry

IHC was performed using the automatic immunostain system (Ventana Discovery). For the primary antibodies, anti-human mouse monoclonal antibody of CTH (Abnova, clone number MO3) were used. TMAs were stained with diaminobenzidine (DAB) by a standard enzyme-labeled antibody method.

### Construction of the Virtual Pathology

A triple fluorescence filter of the Virtual Slide Scanner (NanoZoomer 2.0-HT, Hamamatsu Photonics KK, Hamamatsu, Japan) was used to scan the IF sections. Images of the virtual pathology that were created were constructed by scanner software (Nanozoomer Digital Pathology Virtual Slide Viewer version 2.2, Hamamatsu Photonics KK).

### Automated Quantitative Screening of Protein Expression by Virtual Immunofluorescence Pathology

In order to quantify the protein expression detected by IF of the TMAs, a novel automated quantification system of protein expression from virtual immunofluorescence pathology (AQVIP) images was established using computer software.

### Western Blot Analysis

Cells were extracted with lysis buffer (10 mmol/L HEPES (pH7.4), 150 nmol/L NaCl, 1 mmol/L EDTA, 1% Triton X-100, 1% NP40, and 1 mg/ml NaN3) containing a protease inhibitor cocktail (Sigma-Aldrich, St. Louis, Mo.) on ice for 30 min. Cell lysates were separated by SDS-PAGE and transferred to Immobilon-P membranes (Millipore, Billerica, Mass.). After incubation with primary antibodies at 4° C. overnight and with the relevant secondary antibodies at room temperature for 1 h next day, the reaction was finally detected with enhanced chemiluminescence western blotting detection reagents (GE Healthcare, Pittsburgh, Pa.).

### Drug and Growth-Inhibitor Assay

Cysplatin (CDDP, Randa, molecular weight, 300.5 Da) was purchased from Nippon Kayaku (Tokyo, Japan). Propargylglycine (PPG, molecular weight, 113.11 Da), a specific inhibitor of CTH, was purchased from Sigma-Aldrich. OVISE, OVTOKO, and OVCAR-3 were seeded into opaque-walled 96-well plates in a concentration of 1×10⁴ cells/well on the day before adding the drugs. CDDP solution of various concentrations (0, 0.02, 0.1, 0.5, 2.5, and 12.5 g/ml) was then added to the wells in duplicate. In the following, PPG dissolved in water (0, 0.1, 0.5, 1.0, 5.0, 10 g/ml) was added. 96 hours after addition of the drugs, cell viability was quantified using the Cell Titer-Glo Luminescent Cell Viability Assay (Promega, Madison, Wis.). Luminescence activity was measured with a GloMAx96 Microplate Luminometer (Promega).

### Statistical Analysis

The adequate cut-off point for outcome prediction of ovarian cancer patients was determined by X-tile analysis, a bio-informatics tool for biomarker discovery. Patients were classified into two groups, either positive group or negative group. Survival curves were plotted using the Kaplan-Meier method starting from the date of surgery to the date of death or last follow-up with stratified log-rank test (R-project). Chi-square test, Fisher exact test, Student t-test and the Cox proportional hazards regression model were performed with the StatFlex statistics package (version 5.0; Atiteck, Osaka, Japan) and the R-Project.

### Example 2

### Results

### Establishment of AQVIP

The quantification of the tissue microarray (TMA) was performed by taking the following steps. First, the black rectangular array template, on which white circles of spot templates were allocated at even intervals, was matched on the image of the TMA. Second, the position of each spot template on the array template was quarterly adjusted to each spot image, using correlation coefficients between spot images and corresponding spot template images.

Reaction intensity scores were meaning of red fluorescence intensity of cancer tissue areas. To detect cancer tissue areas, which stained by CK19 (green) and create cancer masks, the target spot image was binarized using only the green color component and distinguished white areas as cancer tissue. For the binarization, two types of methods were used, the Otsu's method or the area segmentation method using an image pyramid. The former was the popular binarization method and specifically allowed to strictly distinguish cancer areas from necrotic or intestinal cell areas. However, when green intensity of whole image was low, the cancer areas was sometime missed identified due to the outlier effect of exceedingly low intensity levels of glass areas became relatively higher. To detect those areas, only tissue existing areas exhibited more than 5.0 blue autofluorescence was used for the binarization, and the outlier of exceedingly low intensity levels of black glass areas was eliminated. In the latter, following the area segmentation using four image pyramids (the threshold of the linkage was 255.0, the threshold of clustering was 50.0), images were binarized to divide the darkest segment from other segments. If the maximal intensity of image is lower than the threshold of clustering, the maximal intensity was used as the threshold of clustering to detect cancer areas from low intensity tissue areas. The binary image was modified by filling holes of a particular size (less than 100 pixels) to fill in cancer nuclei and by removing extraneous small areas of a particular size (less than 100 pixels) to eliminate dusts or any errors. Then, the target spot image was masked using the modified binary image, and only cancer tissue areas were extracted. Finally, the reaction intensity score was calculated as the sum of red color component intensity of areas, which stained by antibody of CK-19 (green) and the target antibody (red) reacted to, divided by the cancer area.

### Antibody Screening by Overall Survival

To identify the biomarkers for predictive OS of advanced-stage ovarian cancer, it was begun by screening 1012 mouse monoclonal antibodies on the TMAs from which at least 50 surgical specimens, obtained from NDMC, were arranged. Virtual pathology images were constructed from the data of the TMAs that were double-stained with CK19 and the target antibodies. The antibodies were selected by two criteria: (1) the antigens had a significant difference in OS by log-rank test (p<0.05); and (2) the distinct expression of the antigens could be confirmed in cancer cells from virtual pathology images by the observer. The selected antibodies were examined again on the 139 surgical specimens obtained from NDMC (training cohort-NDMC). Moreover, the antibodies that were selected from the training cohort-NDMC were validated using the validation cohort-NCC. Finally, 12 antibodies were identified as biomarker candidates for predictive OS of advanced-stage of ovarian cancer. Cut-off values were determined using the X-tile algorithm. Cystathionine gamma-lyase (CTH) was highly expressed in patients with poor prognosis.

### Clinicopathological Characteristics of Tumors Expressing the Selected Antigens

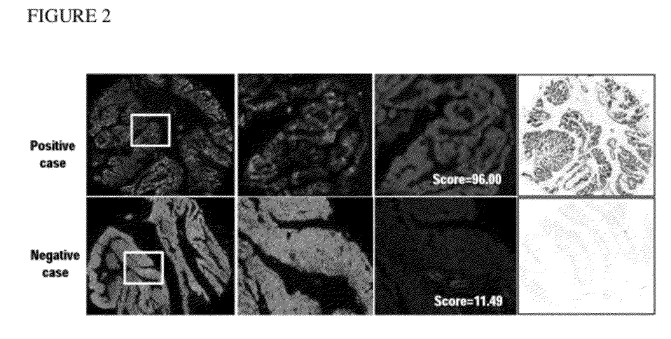

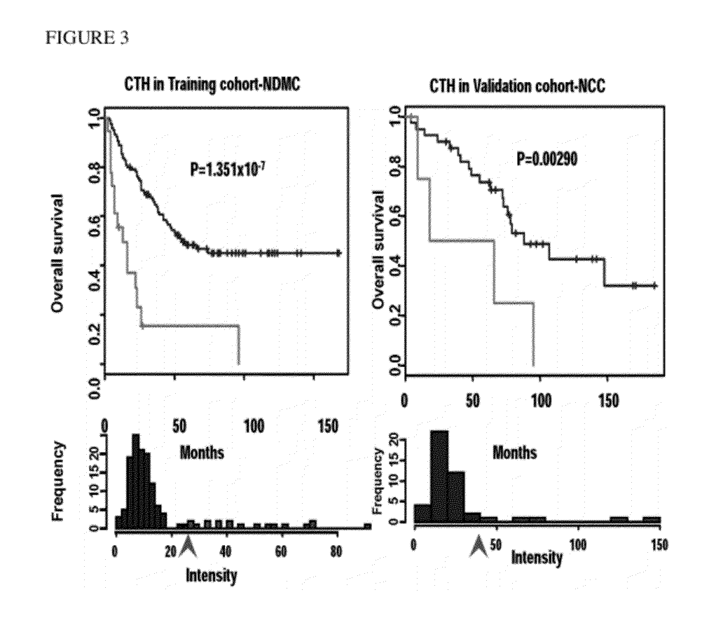

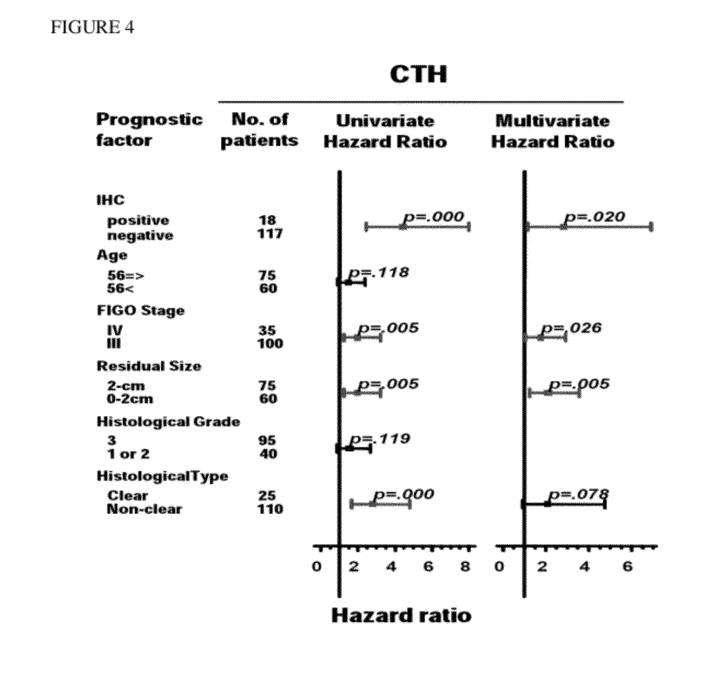

Representative immuno-staining results and quantified score of CTH were shown in FIG. 2. Immunofluorescent virtual pathology confirmed by IHC with DAB was also presented in FIG. 2. Kaplan-Meier curves of CTH in the patients with poor prognosis were shown in FIGS. 3-4.

In the training-cohort-NDMC, the incidence of cases with high CTH expression was 13.3% (18/135) and low expression was 86.7% (117/135). In the validation-cohort-NCC, the incidence of high CTH expression was 9.5% (4/42) and low CTH expression was 90.4% (38/42). The correlation of OS with the high-expression and low-expression cases in both cohorts was statistically significant (training-cohort-NDMC; P=1.35×10⁻⁷, and validation-cohort-NCC; P=0.00290, by log-rank test). The cut-off point of CTH was determined by x-tile analysis, as 26.1.

When the patients with ovarian cancer were classified into positive cases and negative cases by the cut-off point, expression of CTH was statistically associated with the histological types (p=1.40×10⁻¹¹) and efficacy of CDDP-based chemotherapy (p=0.00059) (Table 2).

TABLE 2

| | CTH | | |
|---|---|---|---|
| | Positive | Negative | P = |
| Age | | | |
| 55≧ | 9 | 66 | 0.610385 |
| 56≦ | 9 | 51 | |
| FIGO^{a} stage | | | |
| III | 15 | 85 | 0.402291 |
| IV | 3 | 32 | |
| Histological subtype | | | |
| Serus | 1 | 87 | 1.40E−11 |
| Clear cell | 14 | 11 | |
| Endometrioid | 1 | 9 | |
| Mucinous | 1 | 11 | |
| Histological Grade | | | |
| 1 or 2 | 5 | 39 | 0.7896 |
| 3 | 13 | 78 | |
| Histological grade^{b} | | | |
| 2 cm> | 7 | 53 | 0.799636 |
| 2 cm≦ | 11 | 64 | |
| Response to chemotherapies^{c} | | | |
| CR/PR | 1 | 42 | 0.000593 |
| SD/PD | 10 | 22 | |

^{a}International Federation of Gynecology and Obsterics
^{b}Silberberg's grading
^{c}Patients with residual tumors >2 cm in size after initial surgery

Univariate analyses for hazard risk of death using the Cox proportional hazard model including number of parameters, such as immunoreactivity of CTH, age FIGO stage, the presence of residual tumor size (2 cm), grade, and clear cell adenocarcinoma histology, were analyzed in the training-cohort-NDMC (FIG. 4). Immunoreactivities of CTH correlated with poor patient outcome (P<0.001). The hazard ratio was 4.55 (95% confidence interval 2.22-8.00). Multivariate analysis using the Cox proportional hazard model demonstrated that high expression of CTH independently impacted OS (P<0.001).

Correlation with Response to Chemotherapy

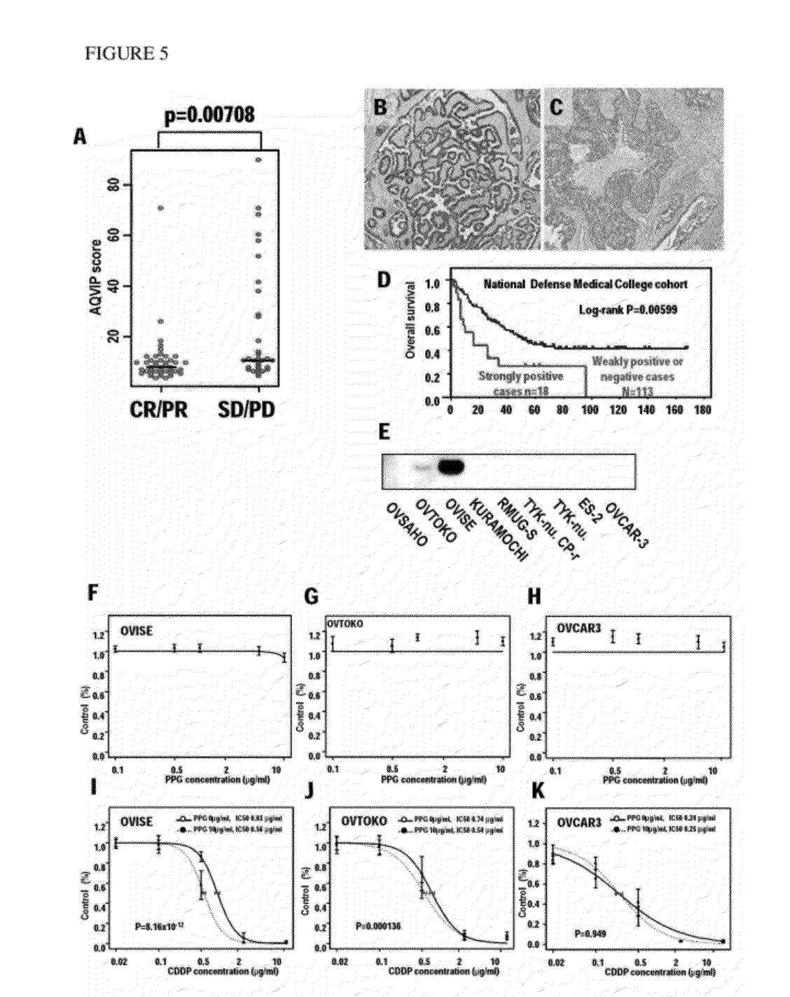

To identify therapeutic targets, the potential of CTH as biomarker for efficacy of CDDP-based chemotherapy was investigated (Table 3). Expression of CTH was significantly different between the responders and the non-responders. Expression of CTH in non-responders was significantly higher than that in the responders (P=0.00708) (FIG. 5A, Table 3).

TABLE 3

| | CR/PR | SD/PD | |
|---|---|---|---|
| Antibody name | (avereage ± SD) | (avereage ± SD)2 | t-test p = |
| CTH(MO3) | 10.32 ± 10.38 | 23.01 ± 23.63 | 0.00708 |

### Validation for Protein Expression of CTH by Immunohistochemistry

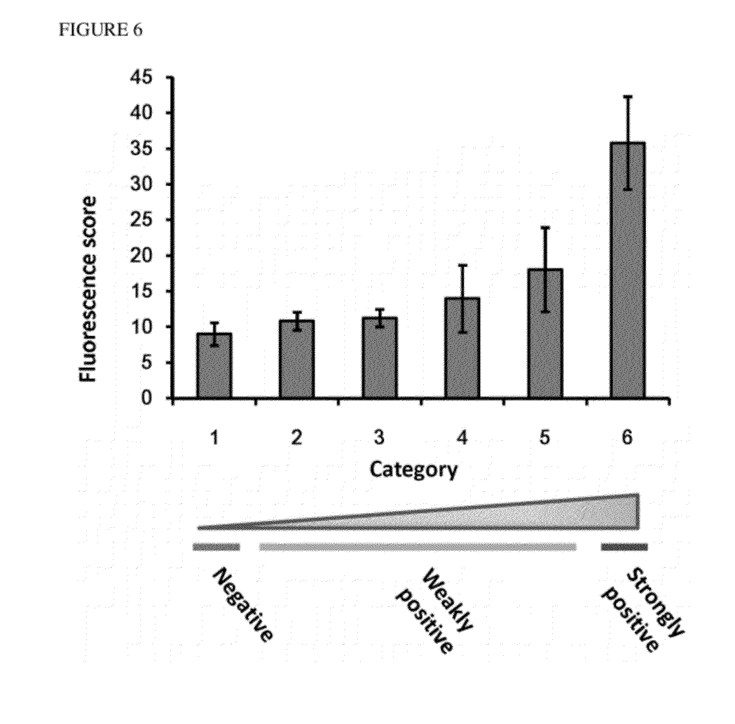

In order to be easily applied in the clinical situation, construction of the evaluation for regular immunohistochemical analysis with DAB was tested. The protein expression of CTH was examined by the immunohistochemistry using DAB in the regular pathological sections from 132 cases of training cohort-NDMC that were examined in first screening Immunohistochemistry result of each case from training cohort-NDMC was blindly evaluated and classified into six categories by a pathologist (FIG. 6). The 6 categories of immunohistochemistry using DAB were correlated with scores of immunofluorescence (FIG. 6). A case would be defined as strongly positive when CTH expression of more than 30% of cancer cells in cancer area was much stronger than that of the smooth muscle (FIGS. 5B and 5C). Strongly positive cases demonstrated poorer outcome for OS than weakly positive or negative cases (FIG. 2A and FIG. 5D) in training cohort-NDMC (Tokorozawa, Japan). The survival curves were closely corresponded to the evaluation of immunofluorescent in training cohort-NDMC (FIG. 5D).

Cysplatin (CDDP) chemosensitivity in the ovarian cancer cell Lines

The protein expression of CTH was examined in 9 ovarian cancer cell lines by western blot analysis. Among all the ovarian cell lines, CTH expression was only observed in OVISE and OVTOKO (FIG. 5E). 50% growth inhibition (IC50) of CDDP was then examined in the OVISE cell line that strongly expressed CTH, in the weak-expressing OVTOKO cell line, and in OVCAR3 cell line which did not express CTH. IC50s of CDDP in OVISE, OVTKO and OVCAR were 0.93, 0.74, and 0.24 g/ml, respectively. There was a positive correlation between protein expression of CTH and IC50 of CDDP in ovarian cancer cell lines (FIG. 5 F-H). The IC50s of propargylglycine, a specific inhibitor of CTH, were also analyzed in these cell lines. Propargylglycine at a concentration ranging from 0.1 g/ml to 10 g/ml had no effect on cell growth of these cell lines. Thus, combination of CDDP and propargylglycine (FIG. 5 I-K) was also examined. When adding 10 g/ml propargylglycine in combination with CDDP into the culture medium, the IC50s was significantly decreased in the CTH-expressed cell lines (i.e., OVISE and OVTKO). However, decrease of IC50 in OVCAR3, which did not express CTH, could not be observed (FIGS. 5I-K).

One skilled in the art readily appreciates that the present invention is well adapted to carry out the objects and obtain the ends and advantages mentioned, as well as those inherent therein. The animals, and processes and methods for producing them are representative of preferred embodiments, are exemplary, and are not intended as limitations on the scope of the invention. Modifications therein and other uses will occur to those skilled in the art. These modifications are encompassed within the spirit of the invention and are defined by the scope of the claims.

What is claimed is:

1. A method of identifying an expression level of cystathionine gamma lyase (CTH) in a sample from a subject comprising:
(a) determining the normal expression level of CTH from a normal subject;
(b) obtaining the sample from the subject; and
(c) determining the expression level of CTH from the sample as over expression if the expression level of CTH from the sample is higher than the normal expression level of CTH; or the expression level of CTH from the sample as normal expression if the expression level of CTH from the sample is equal to the normal expression level of CTH.

2. The method of claim 1, wherein the subject is without pancreatic, bladder, renal, prostate or ovarian cancer.

3. The method of claim 1, wherein the subject suffers pancreatic, bladder, renal, prostate or ovarian cancer.

4. The method of claim 3, wherein the subject has the first biopsy for suspicion of cancer, or is prognosed before or after treated with a platinum-based drug.

5. The method of claim 4, wherein the prognosis is indicated as positive if the sample is CTH over expression.

6. The method of claim 1, wherein the subject suffers pancreatic, bladder, renal, prostate or ovarian cancer and is administered with a platinum-based drug over treatment period.

7. The method of claim 1, wherein the sample is tissue biopsy, body fluid, urine, feces, plasma, serum or blood.

8. The method of claim 1, wherein the level of CTH is determined by immunoassay.

9. The method of claim 8, wherein the immunoassay is western blotting, single-antibody or double-antibody sandwich enzyme-linked immunosorbent assay (ELISA), immunohistochemistry (IHC), immunocytochemistry (ICC) or immunofluorescence (IF).

10. The method of claim 8, wherein the immunoassay involves using a monoclonal or polyclonal antibody against CTH.

11. A method of diagnosing a subject with cancer having risk of resistance to a platinum-based drug comprising:
(a) identifying an expression level of CTH in a sample from the subject with cancer by the method of claim 1; and
(b) determining the subject having risk of resistance to a platinum-based drug if the sample is CTH over expression.

12. The method of claim 11, wherein the cancer is pancreatic, bladder, renal, prostate or ovarian cancer.

13. The method of claim 11, wherein the subject is not administered with the platinum-based drug.

14. The method of claim 11, wherein the platinum-based drug is cisplatin, carboplatin, oxilaplatin, nedaplatin, spiroplatin, iproplatin or satraplatin.

15. A method for improving efficacy of a platinum-based drug in a subject suffering a cancer comprising:
(a) identifying an expression level of CTH in a sample from the subject with cancer by the method of claim 1; and
(b) administering a CTH inhibitor to the subject having risk of resistance to a platinum-based drug if the sample is CTH over expression.

16. The method of claim 15, wherein the cancer is pancreatic, bladder, renal prostate or ovarian cancer.

17. The method of claim 15, wherein the CTH inhibitor is D-propargyl glycine, L-propargyl glycine, DL-propargyl glycine, or salts thereof.

18. The method of claim 15, wherein the administration of the CTH inhibitor to the subject is before, after, or concomitantly with the treatment of the platinum-based drug.
